(19) United States
(12) Patent Application Publication
(10) Pub. No.: US 2026/0335396 A1
(43) Pub. Date: Oct. 29, 2026

(54) Repairing DNA and delaying aging composition, preparation method therefor and application thereof

(71) Applicants: NEWBORN BIOTECHNOLOGY LTD., SURREY (CA); UNQ HONG KONG LIMITED, HONG KONG (CN)

(72) Inventor: YONG WONG, Shanghai (CN)

(73) Assignees: NEWBORN BIOTECHNOLOGY LTD., SURREY (CA); UNQ HONG KONG LIMITED, HONG KONG (CN)

(21) Appl. No.: 19/274,432

(22) Filed: Jul. 18, 2025

(30) Foreign Application Priority Data
Apr. 27, 2025 (CN) 2025105360389

(51) Int. Cl. A61K 36/889; A61K 31/01; A61K 31/122; A61K 31/198; A61K 31/4172; A61K 31/4745; A61K 31/525; A61K 31/714; A61K 36/41; A61P 17/00; A61P 43/00

(52) U.S. Cl. CPC A61K 36/889; A61K 31/01; A61K 31/122; A61K 31/198; A61K 31/4172; A61K 31/4745; A61K 31/525; A61K 31/714; A61K 36/41; A61P 17/00; A61P 43/00

(57) ABSTRACT

A repairing DNA and delaying aging composition, a preparation method therefor and application thereof. The composition comprises following active ingredients in percentage by mass: 10-30% of Rhodiola rosea extract, 5-15% of ergothioneine, 1-5% of pyrroloquinoline quinone (PQQ), 5-20% of acai extract, 0.1-3% of astaxanthin, 0.5-5% of lycopene, 0.1-2% of vitamin B2, 0.01-0.5% of vitamin B12, and the remainder is a pharmaceutically or food-acceptable carrier. The preparation method therefor comprises following steps: step 1, mixing the Rhodiola rosea extract, the ergothioneine, the pyrroloquinoline quinone (PQQ), the acai extract, the astaxanthin, and the lycopene according to mass percentages; step 2, adding vitamin B2 and B12, and homogenizing; and step 3, adding a pharmaceutically or food-acceptable carrier to prepare a target dosage form. The composition achieves an anti-aging effect at the cellular DNA gene level by targeting the DNA repair pathway and combining a multi-component synergistic mechanism.

### CROSS-REFERENCE

This application claims priority to China Patent Application Number 2025105360389 filed Apr. 27, 2025, the contents of which are hereby incorporated by reference in their entirety.

### TECHNICAL FIELD

The present invention relates to the field of biomedicine technology, and in particular to a repairing DNA and delaying aging composition, a preparation method therefor and application thereof.

### BACKGROUND TECHNOLOGY

Current anti-aging technologies mainly rely on single ingredients (such as resveratrol, coenzyme Q10) or simple compounding strategies, focusing on single pathways such as anti-oxidation or improvement of mitochondrial function. Studies have confirmed that low DNA repair efficiency is a core mechanism of aging (Gaspar-Silva F, 2023), but existing technologies have problems such as single targets (such as only activating Sirtuins or inhibiting mTOR), low bioavailability (such as ergothioneine is difficult to penetrate the blood-brain barrier), and lack of adaptation research for the genomic characteristics of East Asian populations.

Among existing technologies, traditional solutions fail to achieve multi-pathway synergy (such as the linkage between DNA repair and energy metabolism), resulting in anti-aging effects limited to local organs (such as improving only the skin or heart); dosage form design does not address ingredient stability (such as PQQ is easily degraded) and delivery efficiency; clinical verification has racial bias (the difference in telomere shortening rate among yellow people is not considered); and safety quality control is insufficient. In addition, existing anti-aging compositions do not cover the construction of cross-organ anti-aging networks and adaptation to specific genotype populations, and cannot meet the needs of precise anti-aging.

### SUMMARY OF THE INVENTION

In view of the shortcomings of the prior art, the purpose of the present invention is to provide a repairing DNA and delaying aging composition, and a preparation method therefor and application thereof, which achieves anti-aging effects at the cellular DNA gene level by targeting the DNA repair pathway and combining a multi-component synergistic mechanism.

The above-mentioned invention purpose of the present invention is achieved through following technical solutions:

On one aspect, the present invention discloses a repairing DNA and delaying aging composition, comprising following active ingredients: Rhodiola rosea extract, ergothioneine, pyrroloquinoline quinone, acai extract, astaxanthin, lycopene, vitamin B2 and vitamin B12.

As a further technical solution of the present invention, the repairing DNA and delaying aging composition, comprises the active ingredients of following mass percentages:
10-30% of the Rhodiola rosea extract, 5-15% of the ergothioneine, 1-5% of the pyrroloquinoline quinone, 5-20% of the acai extract, 0.1-3% of the astaxanthin, 0.5-5% of the lycopene, 0.1-2% of the vitamin B2, and 0.01-0.5% of the vitamin B12, with remainder being a pharmaceutically or food-acceptable carrier.

As a further technical solution of the present invention, the composition is used for activating Sirtuin family proteins (including SIRT1 and SIRT6), inhibiting LOXL2-TGF-β1-Smad2/3 pathway, or enhancing DNA telomerase activity.

As a further technical solution of the present invention, the composition is used for one of following purposes:
repairing DNA damage and improving genome instability;
delaying skin aging and reducing wrinkles, spots and enlarged pores;
improving brain cognitive function and preventing or treating Alzheimer's disease or Parkinson's disease;
protecting heart function and inhibiting myocardial fibrosis or atrial fibrillation; and
repairing lung tissue damage and improving lung inflammation caused by viruses or smoking.

As a further technical solution of the present invention, the composition achieves efficacy through following mechanisms: activating cell-derived life factors to replace damaged DNA fragments, removing free radicals, enhancing mitochondrial function, or promoting differentiation of neural stem cells.

As a further technical solution of the present invention, a dosage form of the composition is capsule, tablet, oral liquid or powder.

On a second aspect, the present invention further discloses a method for preparing a repairing DNA and delaying aging composition, comprising following steps of:
step 1—mixing Rhodiola rosea extract, ergothioneine, pyrroloquinoline quinone, acai extract, astaxanthin, and lycopene according to mass percentages;
step 2—adding vitamin B2 and B12, and homogenizing; and
step 3—adding a pharmaceutically or food-acceptable carrier to prepare a target dosage form.

As a further technical solution of the present invention, in the step 1, the Rhodiola rosea extract is obtained by water extraction or alcohol extraction process, and a total content of flavonoid is not less than 20%.

On a third aspect, the present invention discloses a formulation for improving sequelae of COVID-19, comprising the composition as described above, and further comprising coenzyme Q10 or N-acetylcysteine.

On a fourth aspect, a use of the composition as described above in preparation of a drug for preventing or treating ovarian dysfunction, wherein the composition achieves efficacy by increasing an ovarian AMH index, reducing an FSH level or increasing a number of follicles.

To sum up, the present invention comprises at least one of following beneficial technical effects:

The present invention discloses a composition for repairing DNA and delaying aging, and a preparation method therefor and application thereof. The composition is an anti-aging composition based on the “ELITÉCLAT” gene error correction factor. The composition comprises core ingredients such as Rhodiola rosea extract, ergothioneine, and pyrroloquinoline quinone (PQQ). It significantly improves skin aging, brain cognitive impairment, cardiac dysfunction, and lung damage by activating Sirtuin protein, repairing DNA damage, and scavenging free radicals. It has an anti-photoaging effect on human skin keratinocytes. Experimental data show that it can increase SIRT1 gene expression by 64%, telomerase activity by 70%, and reduce DNA damage by 83%. The composition is suitable for a variety of dosage forms such as capsules and tablets, and has broad application prospects.

I. The present invention uses the synergistic effect of composite active ingredients (Rhodiola rosea, ergothioneine, PQQ, etc.) to target and activate Sirtuin family proteins (such as SIRT1 and SIRT6), repair DNA damage, reduce genomic instability, and enhance telomerase activity (experiments show that telomerase activity is increased by 70%). Its mechanism includes scavenging free radicals, regulating the LOXL2-TGF-β1-Smad2/3 pathway to inhibit fibrosis, and promoting the differentiation of neural stem cells, achieving cell repair and anti-aging at the genetic level.

II. The composition of the present invention shows significant effects in multiple organs such as skin, brain, heart, and lungs: it can improve skin wrinkles (reduced by 21.61% in 28 days) and pigmentation (reduced by 18.44% in 28 days); improve brain cognitive function (immediate memory increased by more than 100%), prevent Alzheimer's disease and Parkinson's disease; repair cardiac fibrosis (reduced probability of atrial fibrillation) and lung damage caused by viruses or smoking; at the same time, it promotes ovarian function (AMH index increased, the number of follicles increased), and assists in repairing the sequelae of the new coronavirus (such as pulmonary fibrosis and nerve damage).

III. The present invention increased SIRT1 gene expression by 64% (p=0.0073) in the zebrafish model, and human clinical studies showed that the collagen regeneration rate increased by 132%. The composition has flexible dosage forms (capsules, tablets, etc.), suitable for anti-aging, disease intervention and health management, and has safety certification (SGS no estrogen residue, heavy metals meet the standards), and has broad medical and consumer application prospects.

### BRIEF DESCRIPTION OF THE DRAWINGS

FIG. 1 is a statistical graph of comet assay data on a DNA damage repair effect of samples in experimental example 1 of the present invention.

FIG. 2 is a statistical graph of comet assay data on a DNA protection effect of samples in experimental example 1 of the present invention.

FIG. 3 is a bar graph of cell viability results of samples in experimental example 2 of the present invention.

FIG. 4 is a bar graph of gene relative expression results in experimental example 2 of the present invention.

FIG. 5 is a comparison chart of an anti-photoaging effect in experimental example 2 of the present invention.

FIG. 6 is a flow chart of a test for promoting expression of zebrafish Sirt1 gene in experimental example 3 of the present invention.

FIG. 7 is a bar graph of relative expression of the zebrafish Sirt1 gene in experimental example 3 of the present invention.

FIG. 8 is a bar graph of relative expression of zebrafish type I collagen gene in experimental example 4 of the present invention.

FIG. 9 is a flow chart of zebrafish aging-related β-galactosidase inhibition test in experimental example 5 of the present invention.

FIG. 10 is a bar graph of anti-galactosidase efficacy test results in experimental example 5 of the present invention.

FIG. 11 is a flow chart of removal test of zebrafish embryo reactive oxygen species (ROS) in experimental example 6 of the present invention.

FIG. 12 is a bar chart of test results of antioxidant efficacy in experimental example 6 of the present invention.

FIG. 13 is a flow chart of zebrafish enhanced telomerase activity test in experimental example 7 of the present invention.

FIG. 14 is a bar chart of zebrafish telomerase activity promotion in experimental example 7 of the present invention.

FIG. 15 is a scatter plot of area ratios of under-eye wrinkles-age in experimental example 8 of the present invention (sample size=200).

### SPECIFIC EMBODIMENTS

The technical solutions in embodiments of the present invention will be clearly and completely described below in conjunction with the drawings in the embodiments of the present invention; obviously, the described embodiments are only some embodiments of the present invention, rather than all the embodiments, and all other embodiments obtained by ordinary technicians in this field based on the embodiments in the present invention without making creative work are within the scope of protection of the present invention.

### Embodiment 1

On one aspect, the present embodiment discloses a repairing DNA and delaying aging composition, comprising following active ingredients: Rhodiola rosea extract, ergothioneine, pyrroloquinoline quinone (PQQ), acai extract, astaxanthin, lycopene, vitamin B2 and vitamin B12.

The active ingredients comprise following mass percentages: 10-30% of the Rhodiola rosea extract, 5-15% of the ergothioneine, 1-5% of the pyrroloquinoline quinone, 5-20% of the acai extract, 0.1-3% of the astaxanthin, 0.5-5% of the lycopene, 0.1-2% of the vitamin B2, and 0.01-0.5% of the vitamin B12, with remainder being a pharmaceutically or food-acceptable carrier. The composition is used to activate Sirtuin family proteins (including SIRT1 and SIRT6), inhibit LOXL2-TGF-β1-Smad2/3 pathway, or enhance DNA telomerase activity.

The composition is used for one of following purposes: repairing DNA damage, improving genome instability; delaying skin aging, reducing wrinkles, spots and enlarged pores; improving brain cognitive function, preventing or treating Alzheimer's disease or Parkinson's disease; protecting heart function, inhibiting myocardial fibrosis or atrial fibrillation; repairing lung tissue damage, and improving lung inflammation caused by viruses or smoking.

The composition achieves its efficacy through the following mechanisms: activating cell-derived life factors to replace damaged DNA fragments, scavenging free radicals, enhancing mitochondrial function, or promoting neural stem cell differentiation. In this embodiment, the dosage form of the composition is one of capsules, tablets, oral liquids or powders.

On a second aspect, the present embodiment further discloses a method for preparing the repairing DNA and delaying aging composition as mentioned above, comprising following steps of:
step 1—mixing Rhodiola rosea extract, ergothioneine, pyrroloquinoline quinone, acai extract, astaxanthin, and lycopene according to mass percentages;
step 2—adding vitamin B2 and B12, and homogenizing; and
step 3—adding a pharmaceutically or food-acceptable carrier to prepare a target dosage form.

In the step 1, the Rhodiola rosea extract is obtained by water extraction or alcohol extraction process, and a total content of flavonoid is not less than 20%.

On a third aspect, the present embodiment further discloses a formulation for improving sequelae of COVID-19, comprising the composition as described above, and further comprising coenzyme Q10 or N-acetylcysteine. And on a fourth aspect, a use of the composition as described above in preparation of a drug for preventing or treating ovarian dysfunction, wherein the composition achieves efficacy by increasing an ovarian AMH index, reducing an FSH level or increasing a number of follicles.

### Embodiment 2

On one aspect, the present embodiment discloses a repairing DNA and delaying aging composition, comprising following active ingredients: Rhodiola rosea extract, ergothioneine, pyrroloquinoline quinone (PQQ), acai extract, astaxanthin, lycopene, vitamin B2 and vitamin B12.

The active ingredients comprise following mass percentages:

10% of the Rhodiola rosea extract, 5% of the ergothioneine, 1% of the pyrroloquinoline quinone, 5% of the acai extract, 0.1% of the astaxanthin, 0.5% of the lycopene, 0.1% of the vitamin B2, and 0.01% of the vitamin B12, with remainder being a pharmaceutically or food-acceptable carrier. The composition is used to activate Sirtuin family proteins (including SIRT1 and SIRT6), inhibit LOXL2-TGF-β1-Smad2/3 pathway, or enhance DNA telomerase activity.

The composition is used for one of following purposes: repairing DNA damage, improving genome instability; delaying skin aging, reducing wrinkles, spots and enlarged pores; improving brain cognitive function, preventing or treating Alzheimer's disease or Parkinson's disease; protecting heart function, inhibiting myocardial fibrosis or atrial fibrillation; repairing lung tissue damage, and improving lung inflammation caused by viruses or smoking.

The composition achieves its efficacy through following mechanisms: activating cell-derived life factors to replace damaged DNA fragments, scavenging free radicals, enhancing mitochondrial function, or promoting neural stem cell differentiation. In this embodiment, the dosage form of the composition is one of capsules, tablets, oral liquids or powders.

On a second aspect, the present embodiment further discloses a method for preparing a repairing DNA and delaying aging composition, comprising following steps of:
step 1—mixing Rhodiola rosea extract, ergothioneine, pyrroloquinoline quinone, acai extract, astaxanthin, and lycopene according to mass percentages;
step 2—adding vitamin B2 and B12, and homogenizing; and step 3—adding a pharmaceutically or food-acceptable carrier to prepare a target dosage form.

In the step 1, the Rhodiola rosea extract is obtained by water extraction or alcohol extraction process, and a total content of flavonoid is not less than 20%.

On a third aspect, the present embodiment further discloses a formulation for improving sequelae of COVID-19, comprising the composition as described above, and further comprising coenzyme Q10 or N-acetylcysteine. And on a fourth aspect, a use of the composition as described above in preparation of a drug for preventing or treating ovarian dysfunction, wherein the composition achieves efficacy by increasing an ovarian AMH index, reducing an FSH level or increasing a number of follicles.

### Embodiment 3

On one aspect, the present embodiment discloses a repairing DNA and delaying aging composition, comprising following active ingredients: Rhodiola rosea extract, ergothioneine, pyrroloquinoline quinone (PQQ), acai extract, astaxanthin, lycopene, vitamin B2 and vitamin B12.

The active ingredients comprise following mass percentages:

30% of the Rhodiola rosea extract, 15% of the ergothioneine, 5% of the pyrroloquinoline quinone (PQQ), 20% of the acai extract, 3% of the astaxanthin, 5% of the lycopene, 2% of the vitamin B2, and 0.5% of the vitamin B12, with remainder being a pharmaceutically or food-acceptable carrier. The composition is used to activate Sirtuin family proteins (including SIRT1 and SIRT6), inhibit LOXL2-TGF-β1-Smad2/3 pathway, or enhance DNA telomerase activity.

The composition is used for one of following purposes: repairing DNA damage, improving genome instability; delaying skin aging, reducing wrinkles, spots and enlarged pores; improving brain cognitive function, preventing or treating Alzheimer's disease or Parkinson's disease; protecting heart function, inhibiting myocardial fibrosis or atrial fibrillation; repairing lung tissue damage, and improving lung inflammation caused by viruses or smoking.

The composition achieves its efficacy through the following mechanisms: activating cell-derived life factors to replace damaged DNA fragments, scavenging free radicals, enhancing mitochondrial function, or promoting neural stem cell differentiation. In this embodiment, the dosage form of the composition is one of capsules, tablets, oral liquids or powders.

On a second aspect, the present embodiment further discloses a method for preparing a repairing DNA and delaying aging composition, comprising following steps of:
step 1—mixing Rhodiola rosea extract, ergothioneine, pyrroloquinoline quinone (PQQ), acai extract, astaxanthin, and lycopene according to mass percentages; step 2—adding vitamin B2 and B12, and homogenizing; and step 3—adding a pharmaceutically or food-acceptable carrier to prepare a target dosage form.

In the step 1, the Rhodiola rosea extract is obtained by water extraction or alcohol extraction process, and a total content of flavonoid is not less than 20%.

On a third aspect, the present embodiment discloses a formulation for improving sequelae of COVID-19, comprising the composition as described above, and further comprising coenzyme Q10 or N-acetylcysteine. And on a fourth aspect, a use of the composition as described above in preparation of a drug for preventing or treating ovarian dysfunction, wherein the composition achieves efficacy by increasing an ovarian AMH index, reducing an FSH level or increasing a number of follicles.

### Experimental Example 1

The “VANPEARL ERGO-VITALIS” gene capsule is prepared by adopting the formula and preparation method of the present invention, and samples of the gene capsule are used to detect the DNA damage repair and protection efficacy. After detection, under the experimental conditions, the sample “VANPEARL ERGO-VITALIS” gene capsule can significantly improve the comet tail length, tail moment and Olive tail moment of the damaged modeled HepG2 cells: when the HepG2 cells after sample treatment are irradiated by UVA, the comet tail length, tail moment and Olive tail moment of the cells are not significantly different from those of the control group, indicating that the sample has the repair and protection efficacy on DNA.

Method name: comet assay.

Method source: laboratory method.

System and grouping.

Test system: HepG2 cells.

Measured values: comet tail lengths, tail moments, Oive tail moments.

Grouping: control group, injury control group, sample group (low concentration group-embodiment 2, medium concentration group-embodiment 1, high concentration group-embodiment 3).

Principle and method: comet assay, also known as single cell gel electrophoresis assay, is a technique for detecting DNA damage and repair in a single cell. Under the action of an electric field, the DNA of a normal cell appears round on the gel due to its large molecular weight, compact structure and low mobility. When the cell DNA is damaged, such as single-strand or double-strand breaks, the DNA unwinds in an alkaline environment, and the broken DNA fragments migrate in the electric field, forming an image resembling a comet's tail. The degree of DNA damage can be quantified by analyzing parameters such as comet tail length, tail moment (the product of tail length and DNA content in the tail), and Olive tail moment (the product of Olive tail length and DNA content in the tail, and the Olive tail length refers to the distance from the center of the comet head to the center of the comet's tail).

If health products have the effect of promoting DNA damage repair, then the comet assay parameters of the DNA detection data of the treated cells in the model group with DNA damage should be better than those of the untreated group after sample treatment, that is, the values of tail length, tail moment, Olive tail moment, etc. are reduced, indicating that DNA damage has been repaired.

If health products have the effect of promoting DNA protection, then the cells treated with samples are less likely to be damaged when subjected to the same external DNA damage (such as UVA irradiation) than cells not treated with samples.

The comet assay parameters of the sample-treated group should be better than those of the untreated group, that is, the values of tail length, tail moment, Olive tail moment, etc. are reduced, indicating that the DNA protection effect of the cells treated with samples is significant.

If health products have the effect of promoting DNA protection, then cells treated with samples will be less likely to suffer DNA damage when subjected to the same external DNA damage (such as UVA irradiation) than cells not treated with samples.

The comet assay parameters of the sample-treated group should be better than those of the untreated group, that is, the tail length, tail moment, Oiv tail moment and other values will decrease, indicating that the DNA protection effect of cells treated with samples is significant.

The experimental steps are as follows:

### 1. Experimental Grouping:

Normal control group: cells were cultured normally without induction of DNA damage and sample treatment.

Damage control group: cells were treated with 100 μMH2O2 to induce DNA damage, and no sample was added.

Low concentration group: cells were treated with 100 μMH2O2 and then treated with 10 μg/mL sample.

Medium concentration group: cells were treated with 100 μMH2O2 and then treated with 50 μg/mL sample.

High concentration group: cells were treated with 100 μMH2O2 and then treated with 100 μg/mL sample.

### 2. Cell Culture:

(1) Resuscitate HpG2 cells, inoculate them in culture bottles with complete culture medium, and culture them in a 37 °C, 5% CO2 incubator. When the cells are in the logarithmic growth phase, conduct subsequent experiments.
(2) DNA damage induction and sample processing: take cells in the logarithmic growth phase, digest them with trypsin-EDTA digestion solution, and adjust the cell density to 1×106 mL: Add 100 M MH2O2 to each treatment group and incubate at 37 °C for 30 minutes to induce DNA damage: After damage treatment, add sample solutions of corresponding concentrations to the low, medium and high concentration groups of samples, and continue to incubate at 37 °C for 2 hours. Add equal volumes of culture medium to the normal control group and the damage control group, and detect the comet assay data of each group.
(3) DNA protection and sample processing: add sample solutions of corresponding concentrations to the low, medium and high concentration groups of samples, and incubate them at 37 °C for 2 hours: Use UVA to irradiate the low, medium and high concentration groups for 20 min, and detect the comet assay data of the low, medium, high concentration groups and the control group.

### 3. Comet Assay Operation:

(1) Preparation of the first layer of gel: coat the slide with NMA (0.5%) and dry at 4 °C.
(2) Preparation of the second layer of gel: mix 100 μL of cell suspension with 900LLMA (0.7%), quickly drop it on the first layer of gel, cover it with a glass slide, and leave it at 4 °C for 10 minutes to solidify the gel
(3) Cell lysis: place the slide in the cell lysis solution and lyse it at 4 °C in the dark for 1 hour
(4) Unwinding and electrophoresis: transfer the slide to alkaline electrophoresis buffer, unwind for 20 minutes to unwind the DNA, and then electrophoresed at 25V, 300 mA for 20 minutes.
(5) Neutralization: after electrophoresis, place the slide in neutralization buffer and neutralize for 15 minutes. Repeat 3 times.
(6) Staining: stain with EB staining solution for 15 minutes, rinse with distilled water and dry.

### 4. Result Observation and Analysis:

(1) Under a fluorescence microscope, randomly select at least 100 cells from each sample for observation.
(2) Use image analysis software (such as Comet Assay IV software) to measure parameters such as comet tail length, tail moment, and Olive tail moment.

### Experimental Reagents, Consumables, and Instruments are as Follows:

1. Experimental instruments: centrifuge, constant temperature water bath, horizontal electrophoresis tank, electrophoresis instrument, fluorescence microscope, cell culture incubator, slide, cover glass, micropipette, cell counting plate.
2. Experimental consumables and reagents: normal melting point agarose, low melting point agarose, cell lysis solution, alkaline electrophoresis buffer, neutralization buffer, ethidium bromide (EB) staining.
3. Modeling drug: 100 μM H₂O₂ treatment to induce DNA damage.
4. Sample: gene capsule prepared by Example 1, Example 2, and Example 3 of the present invention.

Judgment basis: statistical analysis p<0.05, which is judged to be significantly different.

The test results of the samples on DNA damage repair are as follows:

TABLE 1
Comet assay test results of samples on DNA damage and repair

| | Comet Tail | Tail | Olive Tail |
|---|---|---|---|
| Group | Length (μm) | Moment (μm) | Moment (μm) |
| Normal Control | 9.61 ± 0.44 | 4.63 ± 0.29 | 3.57 ± 0.18 |
| Group | | | |
| Damage Control | 17.24 ± 0.86 | 8.03 ± 0.39 | 7.04 ± 0.31 |
| Group | | | |
| 10 μg/mL Group | 15.17 ± 0.77 | 7.19 ± 0.33 | 6.42 ± 0.27 |
| 50 μg/mL Group | 13.82 ± 0.64 | 6.47 ± 0.29 | 5.21 ± 0.24 |
| 100 μg/mL Group | 11.07 ± 0.53 | 5.13 ± 0.24 | 4.08 ± 0.19 |

TABLE 2
Statistics of efficacy index of samples on DNA damage repair

| | Test | | | | |
|---|---|---|---|---|---|
| | Concentration | | Efficacy | | |
| Test Item | (%) | | (%) | p-value | Test Result |
| Sample's | 10 | μg/mL | 23.23 | <0.05 | Significant |
| Effect on DNA | 50 | μg/mL | 47.81 | <0.01 | Highly |
| Damage | | | | | Significant |
| Repair | 100 | μg/mL | 83.82 | <0.001 | Extremely |
| | | | | | Significant |

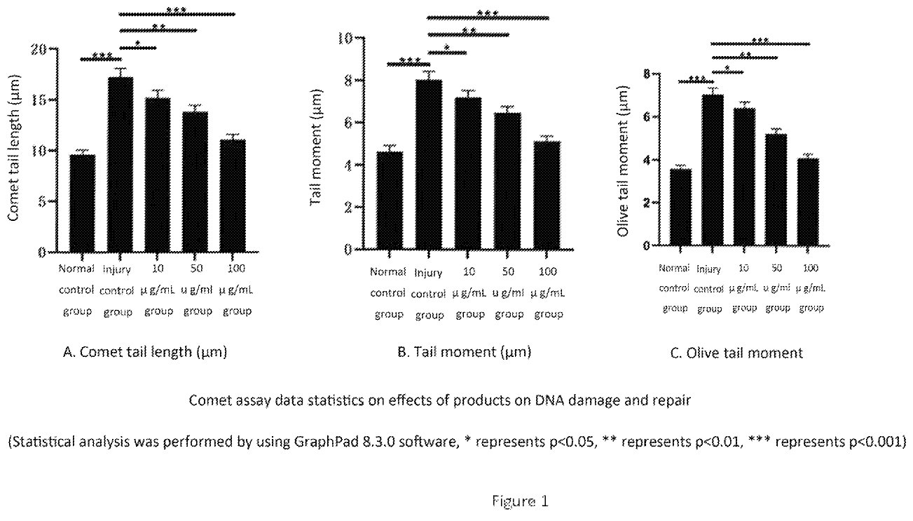

The test results of the DNA damage repair effect of the samples of the present invention are shown in table 1, table 2 and FIG. 1. The results are analyzed as follows: compared with the normal control group, the DNA tail length, tail moment and Olive tail moment of the cells in the damage control group are all lengthened, indicating that the DNA damage model is successfully modeled, and the DNA of HepG2 cells is damaged by H₂O₂ modeling; compared with the damage control group, the DNA tail length, tail moment and Olive tail moment length of the cells in the three concentration treatment groups of the sample group are shortened to varying degrees, and with the increase of the sample treatment concentration, a certain dose dependence is presented, and the data statistics are significant, indicating that the sample has a significant repair effect on the damage caused by the modeling drug H₂O₂ to the DNA of HepG2 cells. Calculated by standardized statistical methods, the sample has a DNA damage repair effect index of 23.23% at a concentration of 10 μg/mL, 47.81% at a concentration of 50 μg/mL, and 83.82% at a concentration of 100 μg/mL.

The test results of the samples' protective effect on DNA are as follows:

TABLE 3
Comet assay test results of the samples' protective effect on DNA

| | Comet Tail | Tail | Olive Tail |
|---|---|---|---|
| Group | Length (μm) | Moment (μm) | Moment (μm) |
| Control Group | 10.24 ± 0.53 | 4.72 ± 0.23 | 3.29 ± 0.16 |
| Control + UVA | 18.33 ± 0.91 | 7.73 ± 0.35 | 6.09 ± 0.31 |
| Irradiation Group | | | |
| 100 μg/mL | 12.11 ± 0.59 | 5.19 ± 0.26 | 4.41 ± 0.22 |
| Treatment Group | | | |
| 100 uμg/mL | 12.32 ± 0.61 | 5.47 ± 0.27 | 4.74 ± 0.24 |
| Treatment + UVA | | | |
| Irradiation Group | | | |

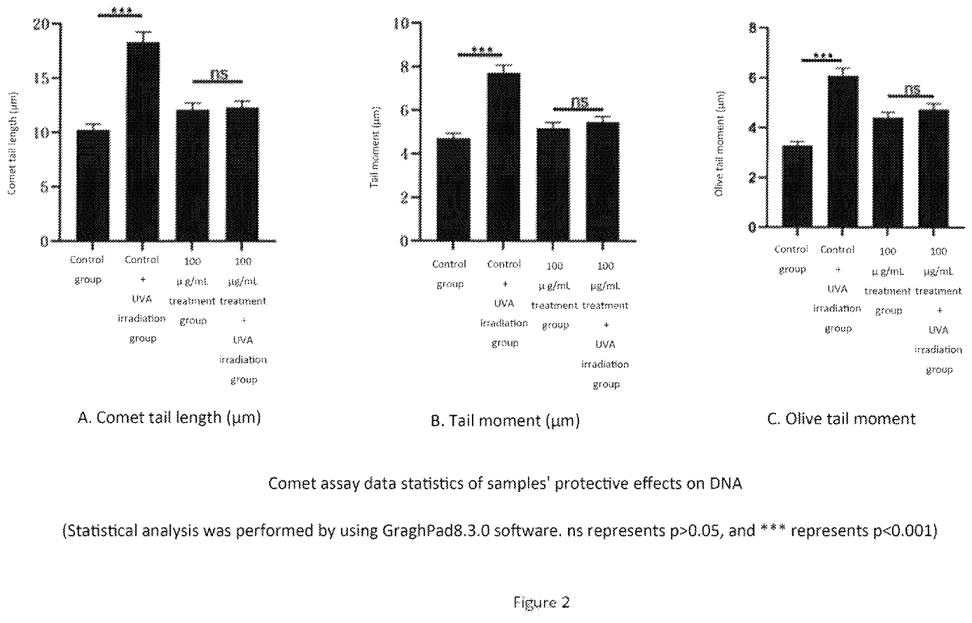

Result analysis: Please refer to FIG. 2 and table 3, compared with the control group, the DNA tail length, tail moment, and Olive tail moment of the cells in the control+UVA irradiation group were significantly longer, and the statistical data showed significant differences, indicating that the DNA of the cells in the control group was significantly damaged after UVA irradiation; compared with the sample treatment group (100 μg/ml), the DNA tail length, tail moment, and Olive tail moment of the cells in the sample treatment+UVA irradiation group did not show a significantly longer phenotype, and the statistical data did not show significant differences, indicating that the sample treatment can protect the DNA of HepG2 cells and reduce the damage caused by UVA irradiation, and has a significant protective effect on cell DNA.

Conclusion: After testing, under the experimental conditions, the sample “VANPEARL ERGO-VITALIS” gene capsule can significantly improve the comet tail length, tail moment and Olive tail moment of the damaged HepG2 cells; the comet tail length, tail moment and Olive tail moment of the HepG2 cells treated with the sample after UVA irradiation were not significantly different from those of the control group, indicating that the sample has the effect of repairing and protecting DNA.

### Experimental Example 2

The “VANPEARL ERGO-VITALIS” gene capsule was prepared using the formula and preparation method of the present invention, and the samples of the gene capsule were used to conduct keratinocyte anti-aging tests.

1. Experimental principle: Repeated exposure to ultraviolet (UV) radiation can cause photoaging of the skin. Depending on the wavelength of UV rays, UV rays enter the skin to varying degrees and interact with skin cells. Among UV rays, UVA (320-400 nm) can penetrate the epidermis to reach the dermis, mainly causing skin photoaging. UVA radiation acts on melanocytes and other skin cells, such as keratinocytes, causing DNA damage through oxidative stress and the production of reactive oxygen species (ROS), which activate signaling pathways related to cell and tissue growth, differentiation, aging, and photoaging. In addition, ROS produced in UVA-irradiated human skin cells are the main cause of photoaging. UV irradiation can also induce the expression of ROS production, leading to skin inflammation and downregulation of SIRT1. SIRT1 is a nuclear protein belonging to the nicotinamide adenine dinucleotide-dependent enzyme family. In addition, SIRT1 plays a key role in regulating cell growth, proliferation, and aging. Therefore, SIRT1 inhibition is generally considered a hallmark of aging, while SIRT1 is involved in UV-induced DNA damage repair, cell metabolism, and photoaging. Increasing evidence supports the hypothesis that photoaging is regulated by a continuous crosstalk between ROS and SIRT1. In this study, human keratinocytes were used as the research subjects, a photoaging model was formed by UVA modeling, SIRT1 and SIRT6 gene expression was detected after the test substances were applied, and the effect of the test substances on anti-photoaging damage was evaluated.

### 2. Experimental Materials:

Experimental samples: “VANPEARL ERGO-VITALIS” gene capsule.

Experimental cells: human immortalized keratinocytes (HaCaT).

Main experimental reagents: DMEM culture medium (LOT: 2537078), fetal bovine serum (LOT: BC20230111), pancreatin (LOT: 2523119), PBS buffer (LOT: 2309011), TRlzol reagent (LOT: 041223230824).

Main experimental instruments: biological safety cabinet (SafeFast Classic 212 A), carbon dioxide incubator (SFEGROW 188 PRO), inverted phase contrast microscope (M152-N), microplate reader (SpectraMax 340PC384), analytical balance (FA2204N), flow cytometer (JIMBIO iCytal S1), cell phototoxicity irradiator (LUYOR-3450), quantitative PCR instrument (ABI PRISM, 7500 Sequence Detection System).

### 3. Experimental Methods:

### 3.1. Cell Viability:

TABLE 4
experimental groups and treatments

| | Experi- | | | |
|---|---|---|---|---|
| Serial | mental | | | Detection |
| Number | Model | Sample/Group | Administration | Index |
| 1 | HaCaT | Zeroing Group | Culture Medium | Cell |
| 2 | | Blank Control | Cells + Culture | Viability |
| | | Group (BC) | Medium | |
| 3 | | Sample | Cells + Different | |
| | | Group (TA) | Concentrations of | |
| | | | Samples in Culture | |
| | | | Medium | |

(1) Cell inoculation: inoculate the cell suspension into a 96-well cell culture plate at 100 μL/well and place it in a carbon dioxide incubator at 37 °C, 5% CO2, and humidity>90% for 18-24 hours.

(2) Drug administration: drug administration was performed according to the experimental grouping and treatment in Table 4. 100 μL of cell culture medium was added to each well of the zero adjustment group and the control group; 100 μL of cell culture medium containing the corresponding concentration of sample was added to each well of the sample group. After drug administration, the 96-well plate was placed in a carbon dioxide incubator at 37 °C, 5% CO2, and humidity>90% for 24 hours.

(3) Cell viability detection: discard the supernatant, add culture medium containing MTT to each well of the blank control group and the sample group, and incubate in a carbon dioxide incubator at 37 °C, 5% CO2, and humidity>90% for 3 hours in the dark. After the incubation, the supernatants of the zero adjustment group, blank control group and sample group were discarded, 100 μL of dimethyl sulfoxide (DMSO) was added to each well, and the absorbance was measured at a wavelength of 570 nm on a microplate reader. The working principle is that the succinate dehydrogenase in the mitochondria of living cells can reduce exogenous MTT to water-insoluble blue-purple crystalline formazan and deposit it in the cells, while dead cells do not have this function. DMSO can dissolve the formazan in the cells, and its light absorption value can be measured with a microplate reader, which can indirectly reflect the number of living cells.

### 3.2 SIRT1 Gene Expression Test:

TABLE 5
experimental groups and treatments

| | | | | Induc- | |
|---|---|---|---|---|---|
| Serial | Experi- | | | tion | Detec- |
| Num- | mental | Sample/ | | Condi- | tion |
| ber | Model | Group | Administration | tion | Index |
| 1 | HaCaT | Blank Control | Cells + Culture | / | SIRT1 |
| | | Group (BC) | Medium | | |
| 2 | | Model Control | Cells + Culture | UVA | |
| | | Group (BC) | Medium | | |
| 3 | | Sample | Cells + Different | | |
| | | Group (TA) | Concentrations of | | |
| | | | Samples in Culture | | |
| | | | Medium | | |

*The blank control group was set up for the purpose of homogenization and calculation of relative gene expression.

(1) Cell inoculation: 2 mL/well of cell suspension was inoculated into a 6-well cell culture plate and placed in a carbon dioxide incubator at 37 °C, 5% CO2, and humidity>90% for 18-24 hours.

(2) Modeling and drug administration: Drug administration was performed according to the experimental grouping and treatment in Table 5. 2 mL of culture medium was added to the blank control group and the model control group, and 2 mL of culture medium containing a certain concentration of the test substance was added to the sample group, and the cells were exposed for 24 hours. After the end, the plate was washed with PBS, and the model control group and the sample group were irradiated with UVA. After the irradiation, the samples of each group were re-prepared according to the drug administration conditions in Table 5 and incubated again for 4 hours.

(3) Gene expression detection: Wash twice with 1 mL PBS. After the washing, 1 mL of Trizol reagent was added to each well of the blank control group, model control group, and sample group to fully lyse the cells. RNA was extracted, reverse transcribed to cDNA, and then fluorescent quantitative PCR was performed. The internal reference was β-actin.

### 3.3 Data Analysis:

(1) Cell Viability Calculation: According to the formula:

$$Cell viability ( \% ) = \frac{Sample group OD - Zeroing group OD}{Blank control group OD - Zeroing group OD} \times 100 \% .$$

(2) Gene Relative Expression Calculation: The relative expression is calculated using the 2-ΔΔCt method with reference genes. The values outputted from the quantitative PCR instrument are Ct values.

ΔCT (Sample Group)=CT (Sample Group)−CT (Experimental Reference).

ΔCT (Blank Control Group)=CT (Blank Control Group)−CT (Control Reference).

ΔΔCT=ΔCT (Sample Group)−ΔCT (Blank Control Group).

Relative gene expression=2^{-ΔΔCt}.

A bar graph was drawn based on the experimental data. The experimental data were homogenized with the blank control group (BC) and statistically analyzed using the t-test method. Compared with the model control group (M), significance is indicated by *, P-value <0.05 is indicated by *, P-value <0.01 is indicated by **, and P-value <0.001 is indicated by ***. If the SIRT1 gene is significantly downregulated in the model control group (M) compared with the blank control group (BC), it means that the experimental modeling is successful; if the SIRT1 gene is significantly upregulated in the sample group (TA) compared with the model control group (M), it means that the sample has an anti-photoaging effect.

### 4. Experimental Results:

(1) Cell viability: 8 dosing concentrations were set for the samples, and cytotoxicity tests were carried out. The MTT test results are shown in Table 6 below.

TABLE 6
sample cell viability results

| | Blank | | | | | | | | |
|---|---|---|---|---|---|---|---|---|---|
| Cell | Control | Concentration Gradient (mg/mL) | | | | | | | |
| viability | Group (BC) | 10.000 | 3.165 | 1.001 | 0.317 | 0.100 | 0.032 | 0.010 | 0.003 |
| Mean | 100.00 | 17.99 | 22.19 | 22.72 | 28.85 | 66.61 | 94.93 | 99.84 | 113.66 |
| SD | 10.86 | 6.33 | 1.20 | 0.89 | 3.89 | 7.29 | 7.53 | 6.11 | 4.84 |

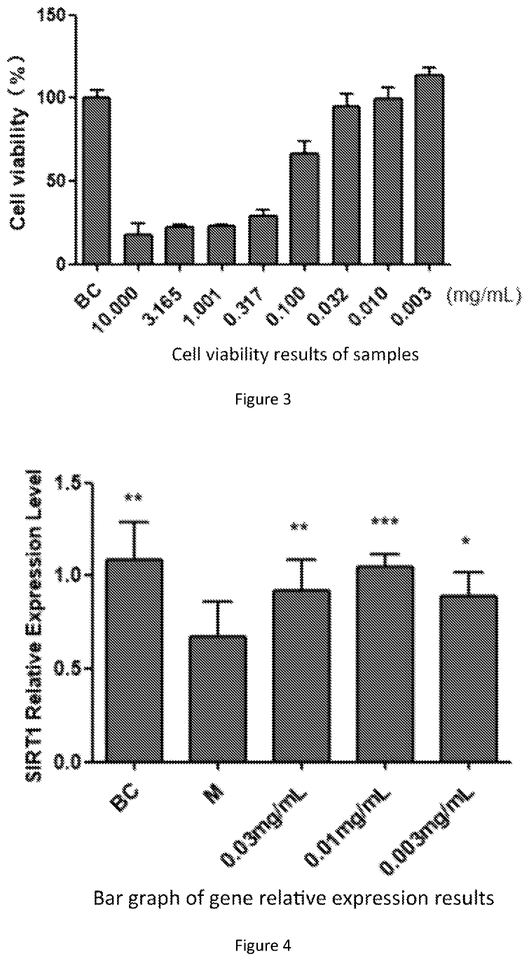

Please refer to FIG. 3, according to the cell viability results, the formal experimental test concentrations were selected as: 0.03 mg/mL, 0.01 mg/mL, and 0.003 mg/mL.

### (2) Gene Expression:

TABLE 7
relative expression of SIRT1 gene in samples

| | | | Relative Gene | | |
|---|---|---|---|---|---|
| | Drug | | Expression | | Enhancement |
| Group | Concentration | | (Mean ± SD) | P-value | Rate |
| BC | / | | 1.08 ± 0.20 | *** | / |
| M | / | | 0.67 ± 0.18 | / | / |
| TA | 0.03 | mg/mL | 0.95 ± 0.16 | ** | 36.89% |
| | 0.01 | mg/mL | 1.05 ± 0.07 | *** | 55.36% |
| | 0.003 | mg/mL | 0.89 ± 0.12 | * | 32.43% |

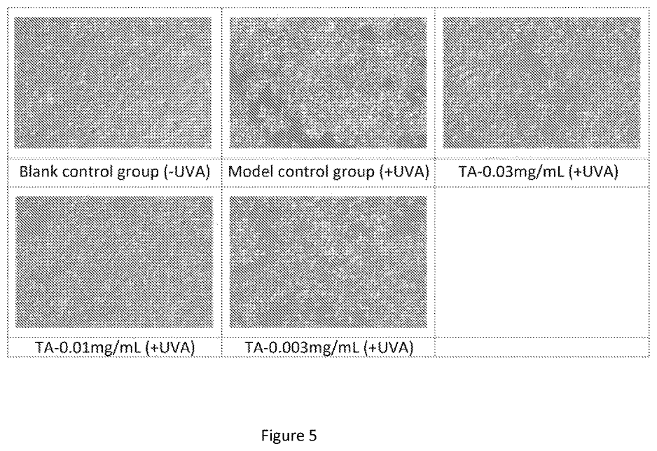

Please refer to Table 7 and FIGS. 4 and 5, the experimental data were statistically analyzed using the t-test method. Compared with the M group, the significance is indicated by *, P-value <0.05 is indicated by *, P-value <0.01 is indicated by **, and P-value <0.001 is indicated by ***.

### 5. Experimental Conclusion:

Under the experimental conditions, compared with the BC group, the relative expression of SIRT1 gene in the M group was significantly downregulated, proving that the photoaging model was successfully established; the relative expression of SIRT1 gene in the sample ergothioneine was significantly upregulated at concentrations of 0.03 mg/mL, 0.01 mg/mL, and 0.003 mg/mL.

In summary, the relative expression of SIRT1 gene was significantly increased by 36.89%, 55.36% and 32.43% at the concentrations of 0.03 mg/mL, 0.01 mg/mL and 0.003 mg/mL of the sample “VANPEARL ERGO-VITALIS” gene capsule, respectively.

In the keratinocyte aging model, the sample “VANPEARL ERGO-VITALIS” gene capsule at the concentrations of 0.03 mg/mL, 0.01 mg/ml and 0.003 mg/m, showed that it could participate in the DNA repair mechanism through the SIRT1 pathway and had an anti-photoaging effect.

### Experimental Example 3

The “VANPEARL ERGO-VITALIS” gene capsule was prepared using the formula and preparation method of the present invention, and the samples of the gene capsule were used for anti-aging efficacy testing, and the zebrafish Sirt1 gene expression promotion test method was used.

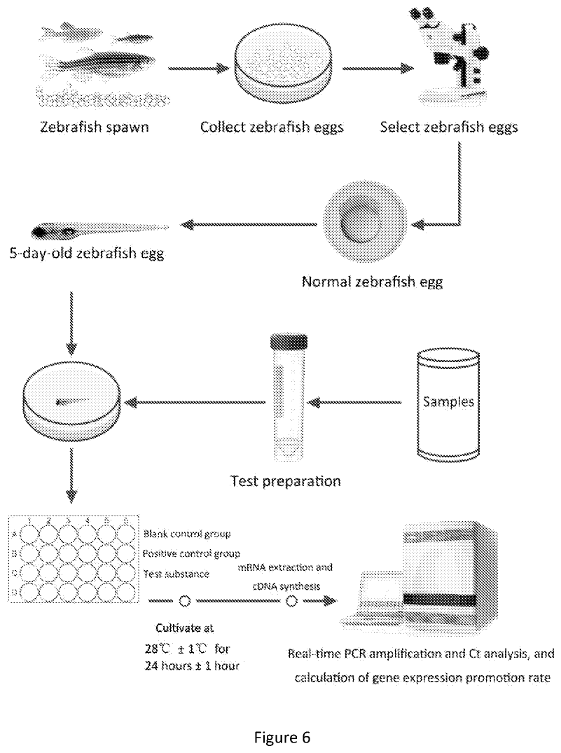

The test method is: Please refer to FIG. 6, 36 5-day-old zebrafish were divided into 3 groups and exposed to 0.0049 g/L gene capsule sample solution, and a blank control group was set up at the same time. After 24 hours of exposure, RNA was extracted from the zebrafish, cDNA was synthesized, and real-time PCR amplification was performed. β-actin was used as the housekeeping gene, and Ct was used as the amplification result. The relative expression of Sirt1 was calculated and statistically analyzed.

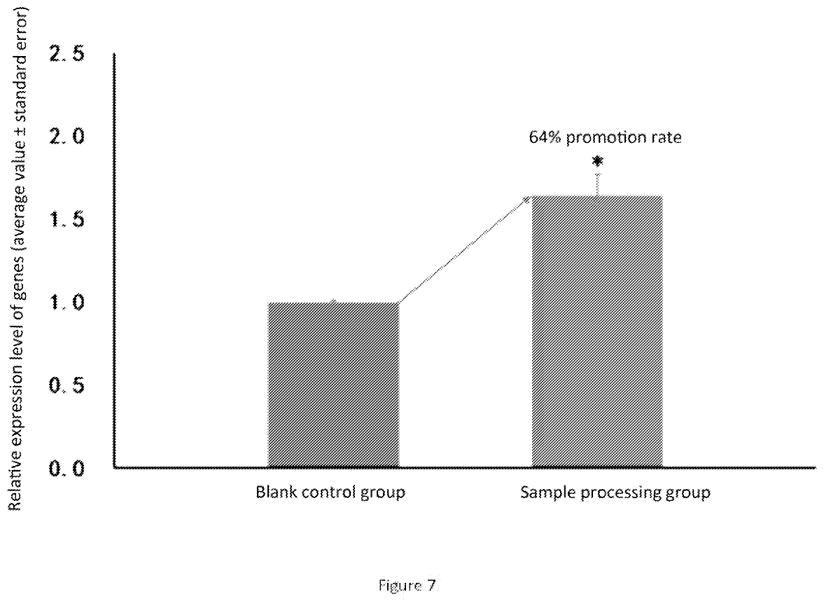

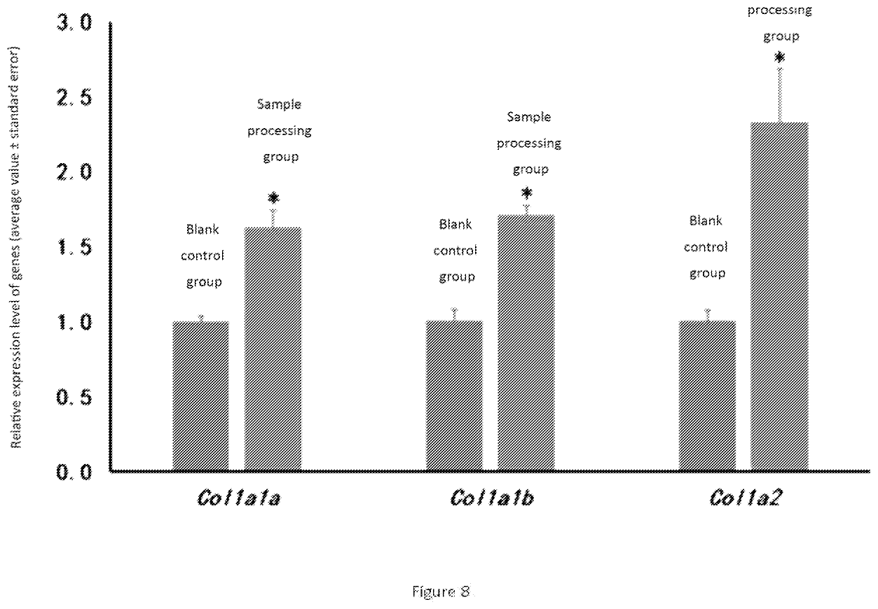

Test results: Please refer to FIG. 7, the sample promoted the expression of zebrafish Sirt1 gene by 64% (p=0.0073) at a test concentration of 0.0049 g/L, and the anti-aging effect was significant.

Conclusion: The sample can significantly promote the expression of zebrafish Sirt1 gene and has an anti-aging effect.

### Experimental Example 4

The “VANPEARL ERGO-VITALIS” gene capsule was prepared using the formula and preparation method of the present invention, and the sample of the gene capsule was used to test the efficacy of promoting collagen, using the zebrafish type I collagen gene expression promotion test method.

The test method is: 36 6-day-old zebrafish were divided into 3 groups and exposed to 0.0049 g/L gene capsule sample solution, and a blank control group was set up at the same time. After 24 hours of exposure, RNA was extracted from the zebrafish, cDNA was synthesized, and real-time PCR amplification was performed. β-actin was used as the housekeeping gene, Ct was used as the amplification result, and the relative expression of col1a1a, col1a1b and col1a2 was calculated and statistically analyzed.

Test results: please refer to FIG. 8, the sample at a test concentration of 0.0049 g/L promoted the expression of zebrafish col1a1a, col1a1b and col1a2 genes by 62% (p=0.0072), 70% (p=0.0021), and 132% (p=0.022), respectively.

Conclusion: the sample can significantly promote the expression of zebrafish type I col1agen gene and has the effect of promoting the regeneration of type I col1agen.

### Experimental Example 5

The “VANPEARL ERGO-VITALIS” gene capsule was prepared using the formula and preparation method of the present invention, and the anti-β-galactosidase efficacy test was performed using the sample of the gene capsule, and the zebrafish embryo aging-related β-galactosidase inhibition test method was adopted.

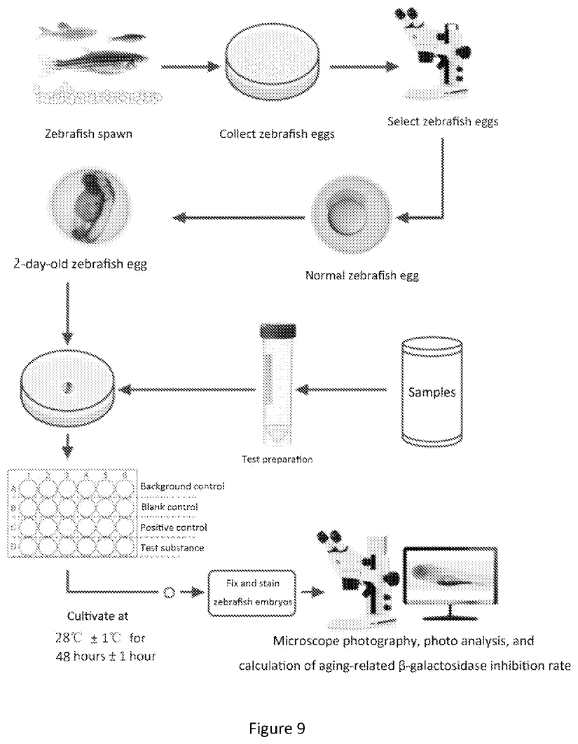

The test method is: please refer to FIG. 9, 24 2-day zebrafish embryos were exposed to 0.099 g/L gene capsule sample solution, and a blank control group and a positive control group were set up at the same time. After exposure for 48 hours, the zebrafish were fixed for aging-related β-galactosidase staining, and the zebrafish aging-related β-galactosidase level was measured under a microscope and calculated and analyzed.

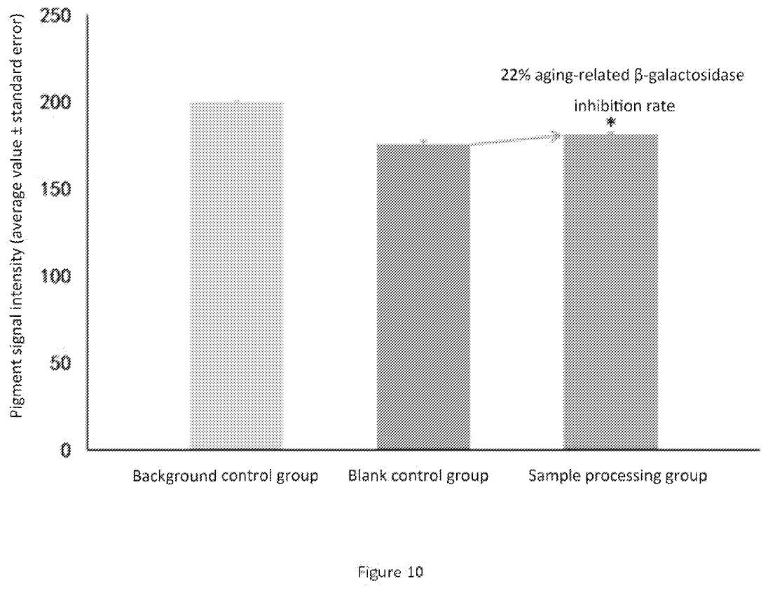

Test results: please refer to FIG. 10, the sample had an inhibition rate of 22% (p=0.0086) on zebrafish embryo aging-related β-galactosidase at a test concentration of 0.099 g/L.

Conclusion: The sample can significantly inhibit the formation of zebrafish embryo aging-related β-galactosidase, and has the effect of inhibiting aging-related β-galactosidase.

### Experimental Example 6

The “VANPEARL ERGO-VITALIS” gene capsule was prepared using the formula and preparation method of the present invention, and the sample of the gene capsule was used for antioxidant efficacy test, and the zebrafish embryo reactive oxygen species (ROS) scavenging test method was adopted.

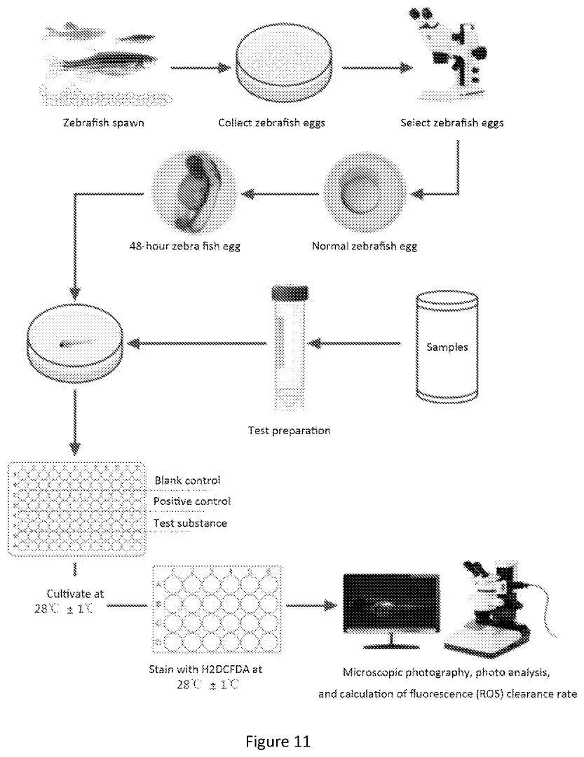

The test method is: Please refer to FIG. 11, 24 48 h zebrafish embryos were exposed to 0.099 g/L gene capsule sample solution, and a blank control group was set up at the same time. After 24 h of exposure, the fish embryos were stained with H2DCFDA, and the ROS signal intensity was measured by fluorescence photography and statistical analysis was performed.

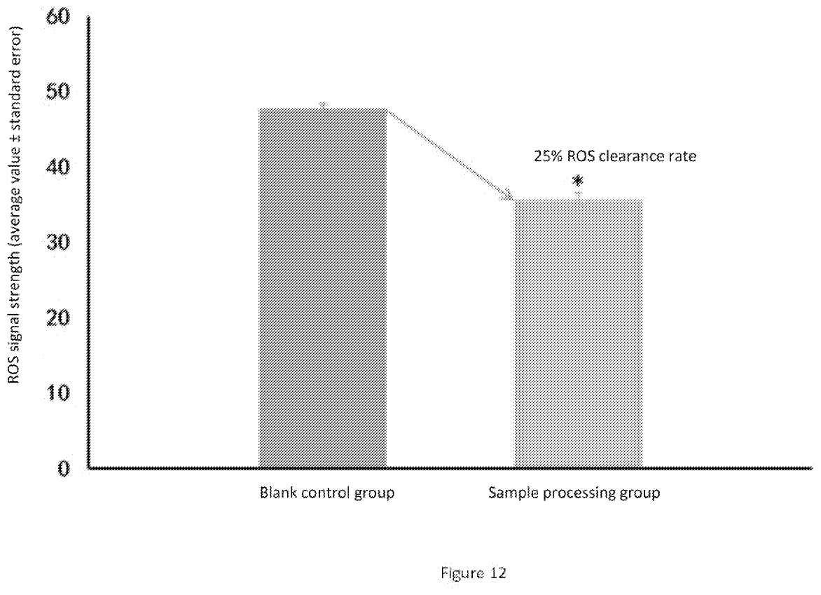

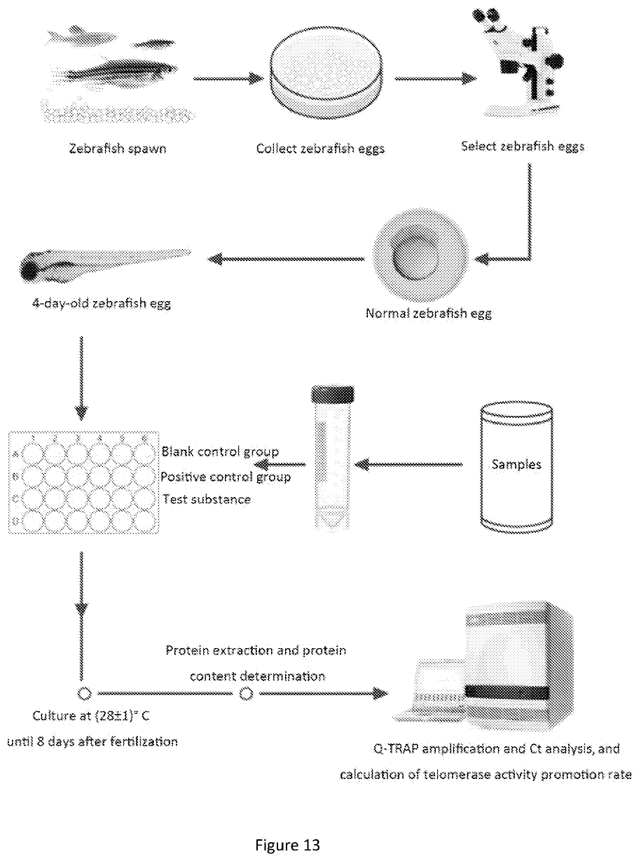

Test results: please refer to FIG. 12, the sample had a ROS scavenging rate of 25% (p=0.000000000018) for zebrafish embryos at a test concentration of 0.099 g/L.

Conclusion: This sample can significantly remove ROS in zebrafish embryos, has an antioxidant effect, and helps with antioxidant effects.

### Experimental Example 7

The “VANPEARL ERGO-VITALIS” gene capsule was prepared using the formula and preparation method of the present invention, and the sample of the gene capsule was used to test the efficacy of enhancing telomerase activity, and the zebrafish telomerase activity promotion test method was used.

The test method is: please refer to FIG. 13, 36 4-day-old zebrafish were divided into 3 groups and exposed to 0.0099 g/L gene capsule sample solution, and a blank control group and a positive control group were set up at the same time. After 4 days of exposure, the zebrafish were subjected to protein extraction, protein content determination, Q-TRAP amplification, and Ct was used as the amplification result to calculate the relative activity of telomerase and perform statistical analysis.

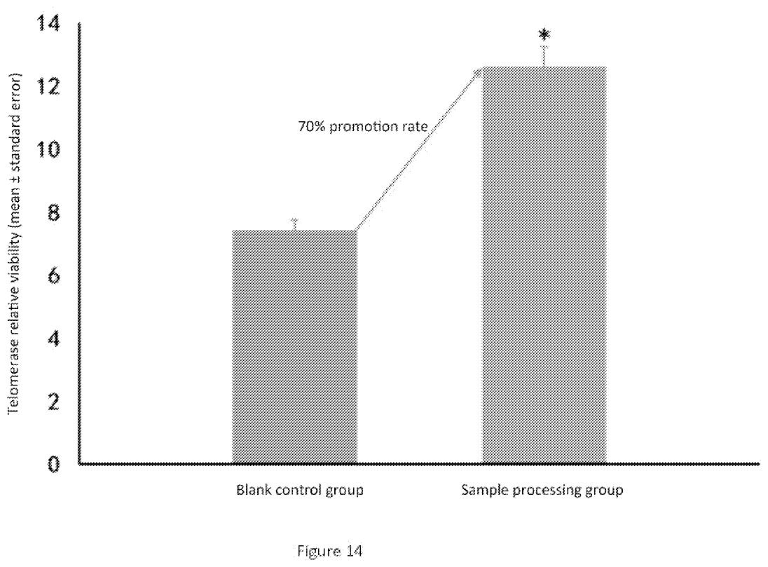

Test results: Please refer to FIG. 14, the sample promoted the telomerase activity in zebrafish at a test concentration of 0.0099 g/L by 70% (p=0.00063).

Conclusion: The sample can significantly promote the telomerase activity in zebrafish and has the effect of enhancing telomerase activity.

### Experimental Example 8

The “VANPEARL ERGO-VITALIS” gene capsule was prepared using the formula and preparation method of the present invention, and the samples of the gene capsule were used for efficacy testing, using the human efficacy evaluation test/consumer use test method.

### The Test Method is:

1) T/ZHCA 006-2019 Cosmetic Anti-wrinkle Efficacy Test Method.
2) “Cosmetic Safety Technical Specifications” (2015 Edition) Chapter 8 5 (Announcement No. 17 of the State Food and Drug Administration in 2021, Annex 7) Cosmetic Spot Removal.

Whitening Efficacy Test Method 2 Human Open Use Spot Removal and Whitening Efficacy Test Method;
3) Liu Weiyi, Zhou Lin, Zhao Hua. Evaluation of cosmetic efficacy (XIII)—Consumer use test [J]. Daily Chemical Industry, 2021, 51 (06): 485-490.
4) “Cosmetic Safety Technical Specifications” (2015 edition).
5) Cosmetic classification rules and classification catalog.
6) Cosmetic efficacy claim evaluation specifications.

### The Test Process is:

### D0:

1) Subject information registration, complete the understanding and signing of the informed consent form, and the laboratory technician screens the subjects according to the inclusion and exclusion criteria.
2) The subjects clean their faces with the designated cleansing samples and sit in the laboratory at a temperature of 21±1 °C and a humidity of 50±10% RH for 30 minutes.
3) Test subject skin basal values: VISIA image acquisition.
4) The laboratory technician guides the subject to take the sample orally according to the sample oral requirements and oral site, and provides written test precautions and oral instructions.
5) The subject receives the sample and leaves the laboratory.

### D14:

1) The subject cleans the face with the designated cleansing sample and sits in the laboratory at a temperature of 21±1 °C and a humidity of 50±10% RH for 30 minutes.
2) Test the skin data value of the subject after 14 days of oral sample: VISIA image acquisition.
3) The subject fills in the self-assessment questionnaire.

### D28:

1) The subject cleans the face with the designated cleansing sample and sits in the laboratory at a temperature of 21±1 °C and a humidity of 50±10% RH for 30 minutes.
2) Test the skin data value of the subject after 28 days of oral sample: VISIA image acquisition.
3) The subject fills in the self-assessment questionnaire.

Test item: ratio of under-eye wrinkle area, test instrument/method: IPP, test time point: D0/D14/D28, test site: under the eye, parameter description: The smaller the value, the lighter the under-eye wrinkles.

Note: D0 refers to the visit before the subject takes the sample orally, D14 refers to the visit 14 days after the subject takes the sample orally, and D28 refers to the visit 28 days after the subject takes the sample orally.

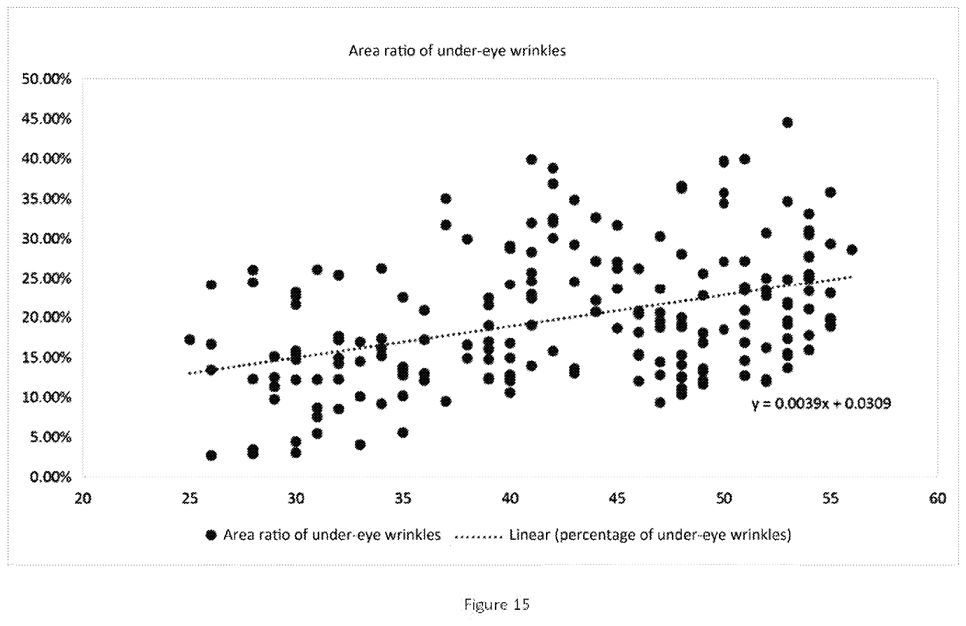

Dimension of under-eye wrinkle area ratio-age value estimation: The test results are shown in FIG. 15 and table 8. In FIG. 15, the samples are selected from 200 subjects aged 25-60 in the subject library; age estimation formula: y=0.0039x+0.0309.

TABLE 8
Age estimation results based on under-
eye wrinkles (area percentage)

| | Estimated Age Result | | | |
|---|---|---|---|---|
| | Under-eye | | | |
| | Wrinkle (Area | Under-eye | Under-eye | |
| | Proportion) | Wrinkle (Area | Wrinkle (Area | |
| | Estimated Age | Proportion) | Proportion) | Average |
| | (Mean ± | Estimated Age | Estimated Age | Age |
| | Standard | (Maximum | (Minimum | Change |
| Test Time | Deviation) | Value) | Value) | (Years) |
| Before Oral | 34.19 ± 3.00 | 40.21 | 26.49 | 1 |
| Intake | | | | |
| 14 Days | 32.53 ± 3.31 | 39.85 | 24.69 | −1.66 |
| After Oral | | | | |
| Sample | | | | |
| 28 Days | 30.87 ± 3.12 | 38.08 | 25.31 | −3.31 |
| After Oral | | | | |
| Sample | | | | |

### Result Description:

Compared with before oral administration, the age value was estimated to be 32.53 years old 14 days after oral administration, which is 1.66 years younger.

Compared with before oral administration, the age value was estimated to be 30.87 years old 28 days after oral administration, which is 3.31 years younger.

The implementation principle of the present invention is as follows: The present invention discloses a composition for repairing DNA and delaying aging, and its preparation method and application. The composition is an anti-aging composition based on the “ELITECLAT” gene error correction factor. The composition contains core ingredients such as Rhodiola rosea extract, ergothioneine, and pyrroloquinoline quinone (PQQ). It significantly improves skin aging, brain cognitive disorders, abnormal heart function, and lung damage by activating Sirtuin protein, repairing DNA damage, and scavenging free radicals. It has an anti-photoaging effect on human skin keratinocytes. Experimental data show that it can increase SIRT1 gene expression by 64%, telomerase activity by 70%, and reduce DNA damage by 83%. The composition is suitable for a variety of dosage forms such as capsules and tablets, and has broad application prospects.

The embodiments of this specific implementation method are all preferred embodiments of the present invention, and are not intended to limit the protection scope of the present invention. Therefore, any equivalent changes made based on the structure, shape, and principle of the present invention should be included in the protection scope of the present invention.

1. A repairing DNA and delaying aging composition, comprising following active ingredients: Rhodiola rosea extract, ergothioneine, pyrroloquinoline quinone, acai extract, astaxanthin, lycopene, vitamin B2 and vitamin B12.

2. The repairing DNA and delaying aging composition according to claim 1, comprising the active ingredients of following mass percentages:
10-30% of the Rhodiola rosea extract, 5-15% of the ergothioneine, 1-5% of the pyrroloquinoline quinone, 5-20% of the acai extract, 0.1-3% of the astaxanthin, 0.5-5% of the lycopene, 0.1-2% of the vitamin B2, and 0.01-0.5% of the vitamin B12, with remainder being a pharmaceutically or food-acceptable carrier.

3. The repairing DNA and delaying aging composition according to claim 1, wherein the composition is used for activating Sirtuin family proteins, inhibiting LOXL2-TGF-β1-Smad2/3 pathway, or enhancing DNA telomerase activity.

4. The repairing DNA and delaying aging composition according to claim 1, wherein the composition is used for one of following purposes:
repairing DNA damage and improving genome instability;
delaying skin aging and reducing wrinkles, spots and enlarged pores;
improving brain cognitive function and preventing or treating Alzheimer's disease or Parkinson's disease;
protecting heart function and inhibiting myocardial fibrosis or atrial fibrillation; and
repairing lung tissue damage and improving lung inflammation caused by viruses or smoking.

5. The repairing DNA and delaying aging composition according to claim 4, wherein the composition achieves efficacy through following mechanisms: activating cell-derived life factors to replace damaged DNA fragments, removing free radicals, enhancing mitochondrial function, or promoting differentiation of neural stem cells.

6. The repairing DNA and delaying aging composition according to claim 1, wherein a dosage form of the composition is capsule, tablet, oral liquid or powder.

7. A method for preparing a repairing DNA and delaying aging composition, comprising following steps of:
step 1—mixing Rhodiola rosea extract, ergothioneine, pyrroloquinoline quinone, acai extract, astaxanthin, and lycopene according to mass percentages;
step 2—adding vitamin B2 and B12, and homogenizing; and
step 3—adding a pharmaceutically or food-acceptable carrier to prepare a target dosage form.

8. The method according to claim 7, wherein in the step 1, the Rhodiola rosea extract is obtained by water extraction or alcohol extraction process, and a total content of flavonoid is not less than 20%.

9. A formulation for improving sequelae of COVID-19, comprising the composition described in claim 1, and further comprising coenzyme Q10 or N-acetylcysteine.

10. A use of the composition according to claim 1 in preparation of a drug for preventing or treating ovarian dysfunction, wherein the composition achieves efficacy by increasing an ovarian AMH index, reducing an FSH level or increasing a number of follicles.
